(19) United States
(12) Patent Application Publication
(10) Pub. No.: US 2007/0117201 A1
(43) Pub. Date: May 24, 2007

(54) Plastic chip for PCR having on-chip polymer valve

(75) Inventors: Soon-Cheol Kweon, Daejeon (KR); Joo Won Rhee, Daejeon (KR); Jun Hyeong Cho, Daejeon (KR)

Correspondence Address: MCKENNA LONG & ALDRIDGE LLP, 1900 K STREET, NW, WASHINGTON, DC, 20006, US

(73) Assignee: LG CHEM, LTD., Seoul (KR)

(21) Appl. No.: 11/589,298

(22) Filed: Oct. 30, 2006

(30) Foreign Application Priority Data
Nov. 18, 2005 (KR) 10-2005-0110789

(51) Int. Cl. C12M 1/34

(52) U.S. Cl. 435/288.3; 435/288.5; 435/305.3

(57) ABSTRACT

The present invention relates to a plastic chip for PCR, having polymer valves, and more particularly to a plastic chip for PCR, having polymer valves, which can perform the temperature cycling of a small amount (nanoliter) of a sample in a microchamber at a constant temperature sensitively to temperature gradient. The inventive plastic PCR chip can perform a PCR even with a small amount of a sample through a reduction in the size of a PCR chamber included in the chip, compared to the prior PCR chip. Also, as the surface area of the PCR chamber is increased, the time taken for heating and cooling can be decreased, thus reducing reaction time. In addition, the inventive plastic PCR chip is mounted with one or more PCR chambers, so that it can amplify various genes at the same time.

### TECHNICAL FIELD

The present invention relates to a plastic chip for PCR, having polymer valves, and more particularly to a plastic chip for PCR, having polymer valves, which can perform temperature cycling of a small amount (nanoliter) of a sample in a microchamber at a constant temperature sensitively to temperature gradient.

### BACKGROUND ART

Technology of amplifying genes has been developed from the use of plastic tubes to a form of chip having microchannels. For the reason of identifying various kinds of genes within a short time at the same time, gene amplification on microchips has been frequently attempted on silicon substrates (Lagally, E. T. et al., Lab on a Chip, 1:102, 2001) or chips made of PDMS (Hong, J. W. et al., Electrophoresis, 22:328, 2001) using semiconductor fabrication processes. This method significantly reduces the amount of sample used and total amplification time, compared to a method of using plastic tubes, but has a shortcoming in that, because it uses semiconductor fabrication processes, the cost efficiency of chip processing is too low to be used as disposable chips. To overcome this shortcoming, a technology of forming microchannels from plastic polymers and using the formed microchannels in gene amplification was developed (Koh, C. G. et al., Analytical Chemistry, 75:4591, 2003).

However, in the case of using these microchannels, there is a problem in that a reaction solution in a chamber evaporates or leaks during PCR reaction due to high temperature. To overcome this problem, the development of a valve capable of effectively opening and closing the microchannel has been required.

Methods for opening and closing microchannels, which have been developed till now, include: a method of using a diaphragm valve, in which force is applied directly to the diaphragm using electromagnetic fields, heat, air pressure piezoelectric elements or the like to bend the diaphragm, such that the bent diaphragm closes the inlet and outlet of a microchannel (U.S. Pat. No. 6,168,948); and a method of using a microfluidic valve, in which a polymer substance showing a phase change depending on temperature is fixed to a microchannel, and the viscosity of liquid is increased using an external heater so as to induce the sol-gel transition of the liquid, so that the biochemical fluid before PCR temperature cycling is confined in a portion of the microchannel (U.S. Pat. No. 6,382,254 and Korean Patent Publication No. 2004-0075217). However, these opening and closing methods are complicated and uneconomical due to the use of electromagnetic fields, heat, piezoelectric elements or the like, and the sol-gel transition method has a shortcoming in that it cannot completely prevent the evaporation and leakage of the reaction solution.

Accordingly, the present inventors have made extensive efforts to develop a method capable of effectively preventing the evaporation and leakage of a solution on a chip and, as a result, found that the use of a polymer membrane deformable by external pressure could effectively prevent the evaporation and leakage of the solution in a PCR reaction. On the basis of this finding, the present inventors have developed a plastic chip for PCR, having polymer valves, thereby completing the present invention.

### SUMMARY OF THE INVENTION

An object of the present invention is to provide a plastic PCR chip having on-chip polymer valves.

To achieve the above object, the present invention provides a plastic chip for PCR, comprising: (a) a microfluidic channel-type reaction chamber formed by depositing and pressing a plastic chip having two or more microfluidic channels, the reaction chamber having an inlet and an outlet; and (b) a polymer valve mounted at each of the inlet and outlet of the reaction chamber, such that it opens and closes fluid flow in the microfluidic channels by external pressure.

In the present invention, the external pressure is preferably applied by a pneumatic cylinder, and an external film heater for temperature cycling is preferably pressed on the surface of the reaction chamber.

In the present invention, the polymer valve is preferably in the form of a polymer membrane. Also, the polymer membrane is preferably made of a material which has good elasticity and can be cast into various shapes. More preferably, the polymer membrane is made of polydimethysiloxane (PDMS).

In the present invention, said plastic is preferably an injectable, extrudable or etchable polymer. Moreover, it is preferably made of any one material selected from the group consisting of polycarnonate (PC), a cyclic olefin copolymer (COC) and polymetamethylacrylate (PMMA), and the plastic chip for PCR comprises two or more reaction chambers.

Other features and embodiments of the present invention will be more fully apparent from the following detailed description and appended claims.

### BRIEF DESCRIPTION OF DRAWINGS

FIG. 1 is a schematic diagram showing a preparation process in which a plastic chip having microfluidic channels is microinjection-molded from a plastic polymer material.

FIG. 2 is a schematic diagram of a plastic chip for PCR according to the present invention.

FIG. 3 is a graphic diagram showing the results of measurement of changes in the temperature of a solution in a microplastic chamber and the temperature of an external microheater during PCR temperature cycling.

FIG. 4 shows the comparison between the results of PCR conducted on a plasmid template using the inventive plastic chip for PCR and the results of PCR conducted using a prior bench top machine.

FIG. 5 shows the comparison between the results of PCR conducted on genomic DNA using the inventive plastic chip for PCR and the results of PCR conducted using the prior bench top machine.

### DETAILED DESCRIPTION OF THE INVENTION, AND PREFERRED EMBODIMENTS

A plastic chip for PCR according to the present invention has a polymer valve membrane for blocking a microchannel (PCR chamber) in order to prevent the evaporation and leakage of a PCR solution at high temperatures. The polymer valve membrane acts as an on-chip valve by pressing the polymer valve membrane using an external pneumatic cylinder.

To conduct temperature cycling using the inventive plastic chip for PCR, an external heater and a cooling fan can be used.

The inventive plastic chip for PCR according to the present invention can comprise two or more microchambers, and thus can amplify two or more genes at the same time.

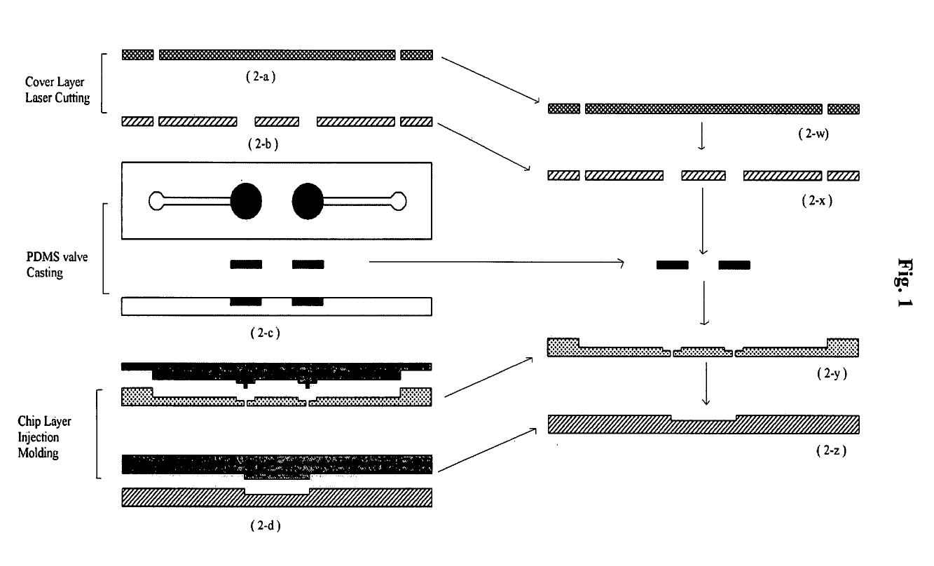

As shown in FIG. 1, the plastic chip having microfluidic channels is fabricated through a microinjection molding process. An injection insert has a positive structure having the same shape as a negative microfluidic channel to be embodied on an injection plastic plane and is prepared by photolithography, micromachining or lapping. When the injection insert is fixed to a mold, and polymer resin is injected in the mold, a plastic sheet having microfluidic channels is then prepared. When the sheet having microfluidic channels is deposited in several layers, which are then adhered to each other by a thermal lamination method or a pressing method using a transparent adhesive, a chip having three-dimensional fluidic chambers and fluidic channels is obtained (FIG. 1).

In the present invention, the plastic sheet is preferably made of a plastic polymer such as polycarnonate (PC), a cyclic olefin copolymer (COC) or polymetamethyl-acrylate (PMMA), which can be used in PCR reactions without special surface treatment.

In the present invention, the polymer valve serves to fluidically open and close the outlet and inlet of the PCR chamber by external pressure and controls the flow of a solution to prevent the leakage and evaporation of biochemical fluid in the PCR chamber.

As the inventive polymer valve, any polymer can be used without limitations, as long as it has waterproofing and heatproofing properties and a property in which it is deformed by external pressure and restored upon the removal of the pressure. Preferably, PDMS can be used.

As shown in FIG. 1, the uppermost layer of the plastic chip for PCR is in the form of a pressure sensitive tape consisting of an adhesive agent coated on a transparent polymer film and covers the uppermost surface of the microfluidic channel. Thus, when the polymer valve is pressed with a pneumatic cylinder, the uppermost layer of the plastic chip for PCR will also be deformed so that it will be involved in opening and closing the polymer valve.

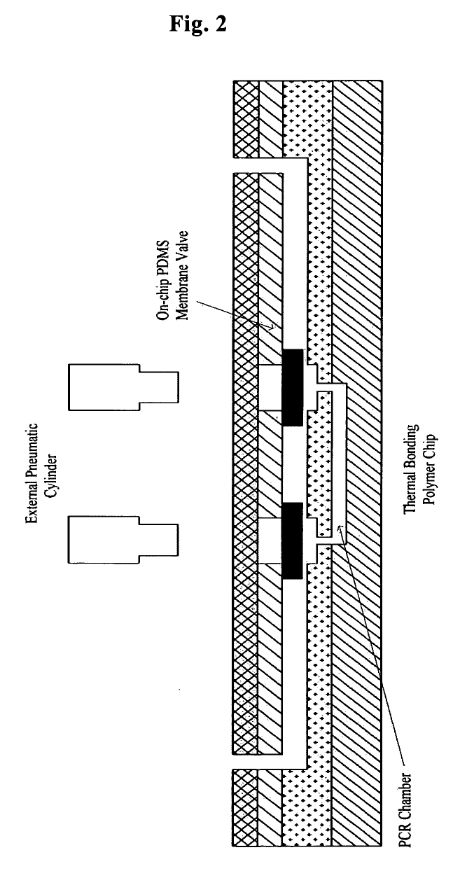

FIG. 2 is a cross-sectional view of a plastic PCR chip mounted with a PDMS on-chip valve, which was finished by thermal lamination and tape lamination after final deposition. In FIG. 1, the intermediate layer and bottom layer of the PCR chamber were bonded with each other using the thermal lamination process, and the two layers were pressed against each other at high pressure. Then, the resulting structure was heated up to about 80% of the glass transition temperature of the polymer material and maintained at that temperature for 1-2 hours, followed by cooling. When the PCR chamber is cycled at PCR temperatures, the microheater is located at the bottom of the PCR chamber to transfer heat upward.

The inventive plastic chip can perform the efficient temperature cycling of a nanoliter of a sample using the microfluidic channels without the loss of a reaction solution.

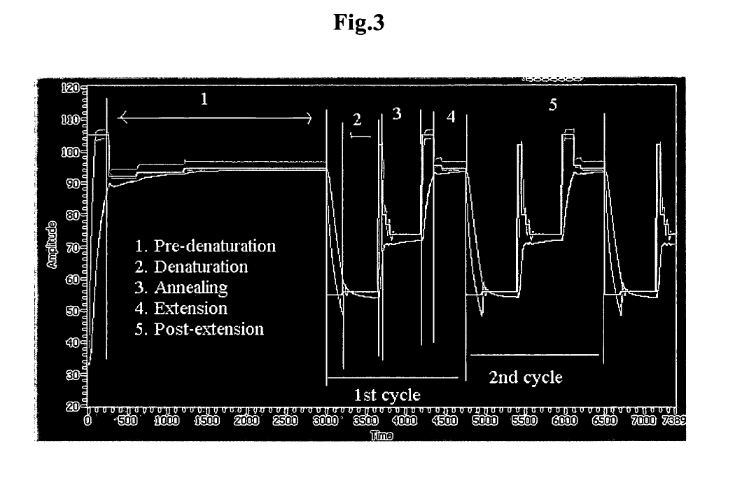

FIG. 3 shows the results of measurement for the temperature of a heater and the temperature inside a PCR chamber during PCR temperature cycling. In the measurements, to overcome the low heat transfer rate of the polymer, the temperature of the heater on a heating curve was rapidly elevated and when the temperature inside the PCR chamber reached set temperature, the set temperature was sequentially lowered. According to this method, among the time required for total PCR, the time required for regions (annealing extension, and extension denaturation) of increasing temperature by heating can be saved.

### EXAMPLES

Hereinafter, the present invention will be described in more detail by specific examples. However, the present invention is not limited to these examples, and it is obvious to those skilled in the field of the present invention that numerous variations or modifications could be made within the spirit and scope of the present invention.

### Example 1

### Fabrication of Plastic Chip for PCR

A plastic chip is fabricated through three steps of cutting of a cover layer, casting of a PDMS valve and injection molding of a chip layer (FIG. 1).

As shown in FIG. 1, the uppermost cover layer 2-w was formed using a polymer film made of the same material as those of a 0.1 mm thick intermediate plastic layer 2-y and a bottom plastic layer 2-z, and the fluid inlet and outlet thereof were formed by laser cutting 2-a. A second layer 2-x serving to bond the uppermost cover layer 2-w with an intermediate plastic layer 2-y was fabricated of a double-sided adhesive tape, which was formed of a silicon-based material and thus maintained strong bonding properties even at high temperatures, and the fluid inlet and outlet thereof were formed by laser cutting 2-b. An intermediate plastic layer 2-y and a bottom plastic layer 2-z were formed by polymer injection molding 2-d. As shown as “2-d ” in FIG. 1, fluid passageways connecting a PCR chamber with another PCR chamber, and inlet and outlet shapes, were machined with an injection insert through metal micromachining and surface polishing, and then the layers were inserted in a mold and injection-molded into a plastic slide chip. The injected intermediate plastic layer 2-y and bottom plastic layer 2-z were bonded with each other by thermal lamination, or were bonded with each other by processing a double-sided adhesive layer such as the second layer 2-x with a laser, inserting the processed adhesive layer between the two layers and then pressing the layers against each other. Generally, the thickness of each of the intermediate layer and the bottom layer is about 1.0-2.0 mm, and the thickness of the PCR chamber is 0.5 mm. Also, a PDMS membrane 2-c was formed in the following manner. A mold having the same shapes as the fluid inlet and outlet of the intermediate layer 2-y was fabricated, and a base solution of PDMS (sylgard@184 silicon, Dow Corning) and a curing agent were mixed with each other at a ratio of 10:1, 5:1, or 3:1, depending on the desired elastic strength. The mixture was poured into the mold 2-c, solidified, detached from the mold and inserted into the fluid inlet and outlet of the injected intermediate layer 2-y. Through said processes, the PDMS membrane valve was combined with the cover layer, the intermediate layer and the bottom layer, thus fabricating a plastic chip which can perform PCR temperature cycling.

### Example 2

### DNA Temperature Cycling Using Inventive Plastic Chip for PCR

A mixture solution for PCR was added into the plastic chip fabricated in Example 1, and an external film heater and an air cooling fan were disposed on the bottom of the PCR chamber. Then, the PDMS on-chip valve was pressed with a pneumatic cylinder to conduct PCR temperature cycling. A thermocouple was attached to the film heater, and another thermocouple was also inserted into the PCR chamber. These thermocouples were used to measure the surface temperature of the film heater and the temperature inside the PCR chamber during the PCR temperature cycling (FIG. 3). As can be seen in FIG. 3, because the heat conductivity of the polymer was similar to the heat conductivity of air, there was an average temperature difference of 4-8° C. due to a PCR chamber thickness of 0.5 mm. In this Example, a chip design was used, in which the film heater was mounted on the outer surface of the PCR chamber, and thus the outer surface of the plastic PCR chamber had no patterned heater. For this reason, a platform for attaching the heater to the outer surface of the PCR chamber was used in order to increase the cost efficiency of the plastic chip.

The plastic PCR chip according to the present invention suitably responded to temperature ranges required for PCR, and each of set time periods, through the temperature cycling system shown in FIG. 3.

### Example 3

### Amplification of Plasmid DNA Using Inventive Plastic PCR Chip

Using the temperature cycling plastic PCR chip of Example 1, mounted with the on-chip polymer valve, and the PCR temperature cycling system of Example 2, comprising the film heater and the cooling fan, a 1-kb fragment of a pET21a vector (Novagen, USA) was amplified with the following primers.

ManhsKAM Nde (forward) primer (SEQ ID NO:1):
5′-AGAGAGCATATGCGTATTTTAACTCAAAATAACCCA-3′
ManhsKAM EcoR (reverse) primer (SEQ ID NO:2):
5′-AGAGAATTCTTAACCGCCGTAAAGGGTCTTATTCGG-3′

The PCR amplification was performed in the following conditions: pre-denaturation for 5 minutes; and then 30 cycles, each consisting of denaturation for 30 sec, annealing for 30 sec and extension for 30 sec; followed by post-extension for 5 minutes (FIG. 3).

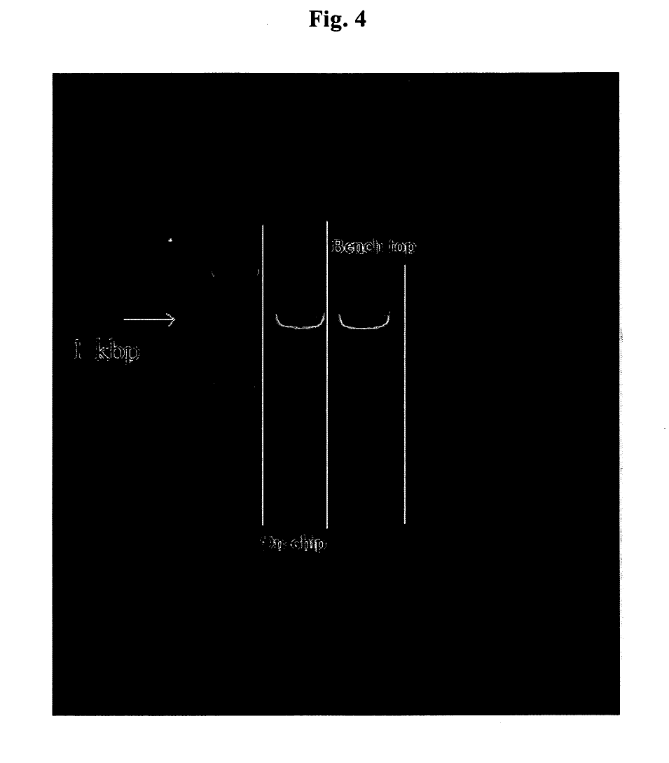

As a result, it could be found that the PCR amplification was made on the inventive plastic chip for PCR at the same level as conducted on the prior bench top machine using tubes (FIG. 4).

### Example 4

### Amplification of Human Genomic DNA Using Inventive Plastic Chip for PCR

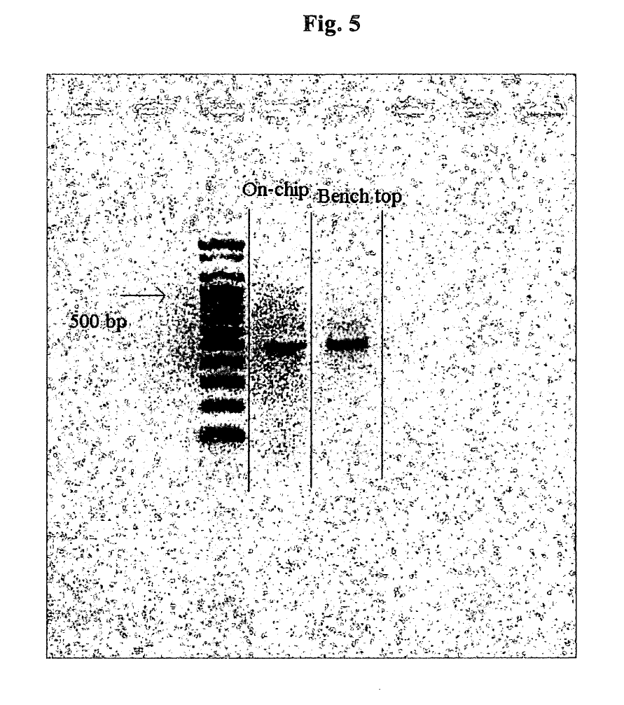

Using the temperature cycling plastic PCR chip of Example 1, mounted with the on-chip polymer valve, and the PCR temperature cycling system of Example 2, comprising the film heater and the cooling fan, the 500-bp beta-actin gene of human genomic DNA was amplified with primers shown in Table 1 below (FIG. 5).

TABLE 1

| | | | PCR |
|---|---|---|---|
| Primers | Sequence | Tm(□) | product |
| Forward | 5′-AAT GAA AGG CAC ACT GTC | 55.3 | beta- |
| primer | TC-3′ | | actin |
| | (SEQ ID NO:3) | | |
| Reverse | 5′-TTT CTT TCA AAG GCT GAG | 55.0 | |
| primer | AG-3′ | | |
| | (SEQ ID NO:4) | | |

In this Example, a genome purified using the blood mini kit (Qiagen, Inc.) was used. The composition of a PCR solution used in the beta-actin amplification reaction using the plastic PCR chip is shown in Table 2 below.

TABLE 2

| Composition of PCR solution | | Volume | Concentration | |
|---|---|---|---|---|
| Forward primer | | 2 μl | 5.0 | nmole |
| Reverse primer | | 2 μl | 5.0 | nmole |
| BSA (Bovine Serum Albumin) | | 2 μl | 0.1 | μg/μl |
| Template DNA | | 2 μl | 80 | ng/μl |
| Distilled water | | 2 μl | | |
| 2× premix | Taq DNA polymerase | 10 μl | 1 units/10 μl | |
| solution | 2× reaction buffer | | — | |
| | MgCl₂ | | 4 | mM |
| | Enzyme stabilizer | | — | |
| | Precipitation reagent | | — | |
| | Loading dye | | — | |
| | dNTPs | 20 μl | each 0.5 | mM |

| | |
|---|---|
| Final volume | 20 μl |

The PCR solution was injected into the plastic PCR chip chamber fabricated in Example 1 and was subjected to PCR using the temperature cycling system shown in Table 3. PCR conditions used in this Example are shown in Table 3 below, and the total time taken for the PCR amplification was 1 hour and 30 minutes, including heating and cooling time.

For use as a control group, the same solution as shown in Table 2 was subjected to PCR in the same conditions as shown in Table 3 below using a bench top PCR cycler (MJ Research, Inc.).

TABLE 3
PCR cycling protocol

| | | |
|---|---|---|
| Pre-denaturation | 5 min | |
| Denaturation | 30 sec | 30 cycle |
| Annealing | 30 sec | 30 cycle |
| Extension | 30 sec | 30 cycle |
| Post-extension | 5 min | |

The reaction solution amplified using the plastic PCR chip in this Example, and the PCR solution amplified using the bench top PCR cycler, were electrophoresed on agarose gel, and the electrophoresis results are shown in FIG. 5. In FIG. 5, lane 1 represents a DNA marker, lane 2 represents the 500-bp genomic DNA gene amplified using the inventive plastic PCR chip, and the lane 3 represents the 500-bp genomic DNA gene amplified using the bench top PCR machine.

As shown in FIG. 5, it was confirmed that the human genomic gene was successfully amplified using the inventive plastic PCR chip, and it is expected that a better PCR efficiency can be obtained through shape deformation and surface optimization for the PCR chamber on the plastic chip.

Although a specific embodiment of the present invention has been described in detail, those skilled in the art will appreciate that this description is merely a preferred embodiment and is not construed to limit the scope of the present invention. Thus, the substantial scope of the present invention will be defined by the accompanying claims and equivalents thereof.

### INDUSTRIAL APPLICABILITY

As described above, the present invention provides the plastic PCR chip having the on-chip polymer valve. The inventive plastic PCR chip can perform PCR even with a small amount of a sample through a reduction in the size of the PCR chamber, compared to the prior PCR chip. Also, as the surface area of the PCR chamber is increased, the time taken for heating and cooling can be decreased, thus reducing reaction time. In addition, the inventive plastic PCR chip is mounted with one or more PCR chambers, so that it can amplify various genes at the same time.

What is claimed is:

1. A plastic chip for PCR, comprising: (a) a microfluidic channel-type reaction chamber formed by depositing and pressing a plastic chip having two or more microfluidic channels, the reaction chamber having an inlet and an outlet; and (b) a polymer valve mounted at each of the inlet and outlet of the reaction chamber, such that it opens and closes fluid flow in the microfluidic channels by external pressure.

2. The plastic chip according to claim 1, wherein the external pressure is applied by a pneumatic cylinder.

3. The plastic chip according to claim 1, wherein an external film heater for temperature cycling is pressed on the surface of the reaction chamber.

4. The plastic chip according to claim 1, wherein the polymer valve is in the form of a polymer membrane.

5. The plastic chip according to claim 4, wherein the polymer membrane is made of polydimethysiloxane (PDMS).

6. The plastic chip according to claim 1, wherein said plastic is an injectable, extrudable or etchable polymer.

7. The plastic chip according to claim 1, wherein said plastic is made of any one material selected from the group consisting of polycarnonate (PC), a cyclic olefin copolymer (COC) and polymetamethylacrylate (PMMA).

8. The plastic chip according to claim 1, which comprises two or more reaction chambers.


SEQUENCE LISTING (4 sequences)

SEQ ID NO: 1 - DNA, 36 bp, Artificial Sequence
  primer
agagagcatatgcgtattttaactcaaaataaccca

SEQ ID NO: 2 - DNA, 36 bp, Artificial Sequence
  primer
agagaattcttaaccgccgtaaagggtcttattcgg

SEQ ID NO: 3 - DNA, 20 bp, Artificial Sequence
  primer
aatgaaaggcacactgtctc

SEQ ID NO: 4 - DNA, 20 bp, Artificial Sequence
  primer
tttctttcaaaggctgagag